(19) United States
(12) Patent Application Publication
(10) Pub. No.: US 2013/0304032 A1
(43) Pub. Date: Nov. 14, 2013

(54) Bone fixation system with structure to enhance tissue growth and/or administer medicament inside bone

(71) Applicant: Neil Rajendra Sardesai, Arcadia, CA (US)

(72) Inventor: Neil Rajendra Sardesai, Arcadia, CA (US)

(21) Appl. No.: 13/875,750

(22) Filed: May 2, 2013

Related U.S. Application Data
(60) Provisional application No. 61/688,161, filed on May 9, 2012.

(51) Int. Cl. A61B 17/86; A61B 17/34

(52) U.S. Cl. CPC A61B 17/864; A61B 17/3472
USPC 604/522; 606/323; 604/93.01

(57) ABSTRACT

A bone fastener or an orthopedic implant that resides within the bone after orthopedic surgery has a threaded and/or unthreaded shaft, said shaft has a hollow portion and a solid portion. The hollow portion may have a structure that enhances tissue growth and may also hold materials that enhance tissue growth. The hollow portion may also hold materials to treat diseases and/or promote bone fracture healing.

### RELATED APPLICATIONS

This patent application claims the benefit of co-pending U.S. Provisional Patent Application Ser. No. U.S. 61/688,161, filed on May 9, 2012, the entire contents of which are incorporated herein in their entirety by reference thereto.

### FIELD OF INVENTION

The present invention generally relates to a bone fastener or an orthopedic implant having a structure that enhances tissue growth and/or having materials that enhance tissue growth, treat diseases and/or promote bone fracture healing.

### BACKGROUND OF THE INVENTION

The present disclosure generally relates to a bone fastener such as a bone screw or an implant such as a hip implant used in orthopedic surgery. The disclosure further relates to an implant system for fixation of bone wherein parts of the implant system such as screws, pegs or rods permanently reside inside the bone after orthopedic surgery or are removed from the bone after a time period in which there is partial or complete bone fracture healing.

Bone screws are available in a plurality of variations for different applications. Other implants such as the femur rod in a total hip replacement system are also available in a plurality of shapes and sizes. U.S. Patent Publication Nos. 2005/0277937, 2005/0261688, 2007/0276386, 2009/0192550 and 2011/0218580 describe screws used to secure a bone plate.

Conventional bone fasteners and implants that reside in the bone do not have structures that promote tissue growth or allow inclusion of medicament for release inside the bone over a period of time after orthopedic surgery.

### SUMMARY OF THE INVENTION

Aspects of the present disclosure are directed to facilitate growth of bone tissue in and around the fasteners and bone implants. The tissue growth is facilitated by the presence of porosities and/or growth factors in a portion of the fastener. Further aspects of the disclosure are directed towards including medicament inside a portion of the fastener to release in the bone mass over a period of time after the surgery.

According to a first aspect, there is provided a bone fastener for use in orthopedic surgery for fixing an implant to bone, wherein the bone fastener comprises a head and a shaft configured to engage bone. The shaft has a hollow portion with openings that allow entry of tissue matter into the hollow portion. The hollow portion also allows for storage or accumulation of materials that facilitate tissue growth. The openings can also be used to introduce materials such as medicaments into the hollow portion before the fastener is inserted into the bone.

The hollow portion can be present for a part or entire length of the shaft. Threads may be present around the hollow portion. The openings can be of various shapes such as round, square, rectangular, triangular, trapezoidal, and polygonal. The sizes of the openings can vary the entire spectrum, with width or diameter of the opening varying from about 1 mm to about the diameter of the shaft, and the height varying from about 1 mm to about the length of the hollow portion.

The head of the fastener can be provided with a hole that communicates with the hollow portion through a channel. The hole can be used to introduce materials in the hollow portion after the fastener is inserted in the bone. The introduction of the material via the channel can be done by suitable methods such as injection, packing, and spraying.

Materials can be introduced in the fastener of the invention which will enhance fibrous growth. The materials can be introduced into the fastener before its insertion into the bone and/or later introduced through the head of the fastener.

Medically/surgically useful materials which promote healing or treatment of a disease condition can also be introduced in the hollow portion of the fastener. Useful materials of this kind which can be introduced into the hollow portion of the fastener include, e.g., collagen, insoluble collagen derivatives and soluble solids and/or liquids dissolved therein, e.g., antiviral agents, particularly those effective against HIV and hepatitis; antimicrobials and/or antibiotics such as erythromycin, bacitracin, neomycin, penicillin, polymyxin B, tetracyclines, chloromycetin and streptomycins, cefazolin, vancomycin, ampicillin, azactam, tobramycin, clindamycin, and gentamicin; amino acids; peptides; vitamins; inorganic elements; co-factors for protein synthesis; endocrine tissue or tissue fragments, synthesizers; enzymes such as collagenase, peptidases, oxidases; polymer cell scaffolds with parenchymal cells; angiogenic drugs and polymeric carriers containing such drugs; collagen lattices; antigenic agents; cartilage fragments, living cells such as chondrocytes, mesenchymal stem cells; natural extracts; genetically engineered living cells or otherwise modified living cells; tissue transplants; bone morphogenic protein; anti-neoplastic agents; DNA delivered by plasmid or viral vectors; autogenous tissues such as blood, serum, soft tissue, bone marrow; bioadhesives; fibronectin; transforming growth factor-beta; endothelial cell growth factors; cementum attachment extracts; ketaserin; insulin-like growth factors (IGF-1)(IGF-2); platelet derived growth factors (PDGF); epidermal growth factor (EGF); interleukin; human alphathrombin; fibroblast growth factors; hormones, in particular, human growth hormone, animal growth hormone and growth hormones such as somatotropin; bone digesters; antitumor agents; immuno-suppressants; permeation enhancers, e.g., fatty acid ester such as laureate, myristate and stearate monoesters of polyethylene glycol, enamine derivatives, alpha-keto aldehydes; and, nucleic acids.

The shaft of the bone fastener may have a core diameter between 1.0 mm and 20.0 mm. The shaft can be unthreaded or at least partially threaded. Generally, a bone fastener having an at least partially threaded shaft can also be referred to as bone screw, whereas a bone fastener with an unthreaded shaft will be referred to as bone peg. The peg may be inserted in the bone using a pilot hole. The bone screw can be a self-tapping screw or a self-drilling screw. Alternatively, the shaft may take the form of an unthreaded pin or rod.

The head can have a constant core diameter. Alternatively, the core of the head can have a conical or curved (e.g., spherical) shape. An outer diameter of the thread of the head may gradually change in a curved (e.g., spherical) or tapering manner.

According to a further aspect there is provided an implant system for use in orthopedic surgery for fixation of bone. The implant system comprises an implant having an upper surface and a lower surface, at least one hole extending through the upper surface and lower surface, and at least one bone fastener. Another aspect of the implant system comprises at least one bone fastener that engages a socket in the implant. In all systems, the at least one bone fastener comprises a shaft configured to engage bone. The head may have a thread on an outer surface to engage the implant.

### BRIEF DESCRIPTION OF THE DRAWINGS

These and other features, aspects and advantages of the present disclosure will become apparent from the following detailed description taken in conjunction with the accompanying drawings, wherein:

FIG. 1 is a view of a first bone fastener embodiment, having at least one hole on the side of the fastener present across the width of the fastener;

FIG. 2 is a cross-sectional view of the first bone fastener embodiment of FIG. 1;

FIG. 3 is a view of a second bone fastener embodiment having at least one cavity on the side of the fastener;

FIG. 4 is a view of a third bone fastener embodiment with at least one valley of the thread portion carved out and hollowed;

FIG. 5 is a view of a fourth bone fastener embodiment with a portion of the shaft hollowed and not threaded;

FIG. 6 is a cross-sectional view of the fastener shown in FIG. 5;

FIG. 7 is a cross-sectional view of the fastener shown in FIG. 1 and having a channel connecting the head to the hollow portion;

FIG. 8 is a cross-sectional view of the fastener shown in FIG. 7, further having a threaded portion to accept a plug to the channel at the head;

FIG. 9 is a cross-sectional view of the fastener shown in FIG. 8 wherein a plug engages the thread in the channel to seal the channel;

### DETAILED DESCRIPTION

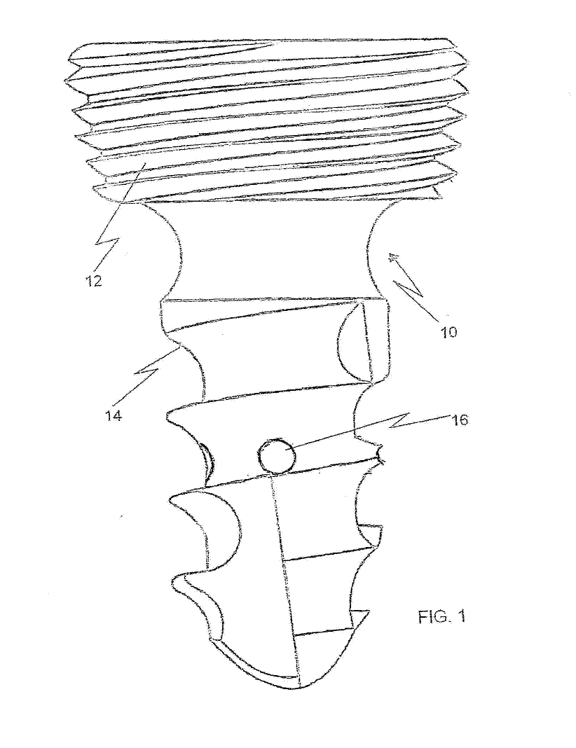

The present invention provides bone fasteners and implant systems, wherein the fastener comprises hollow portion with structure that enhances tissue growth. The hollow portion may have porous metallic structure integral with the screw or may have porous materials added in the hollow portion. The hollow portion may also hold medicament to treat diseases. Referring to FIG. 1, there is shown a side view of a first embodiment of a bone fastener in the form of a bone screw 10 for use as a locking screw in orthopedic surgery for fixing an implant (not shown in FIG. 1) to bone. The bone screw comprises a head 12 and a shaft 14 configured to engage the bone. The head 12 has a thread on an outer surface to engage an implant.

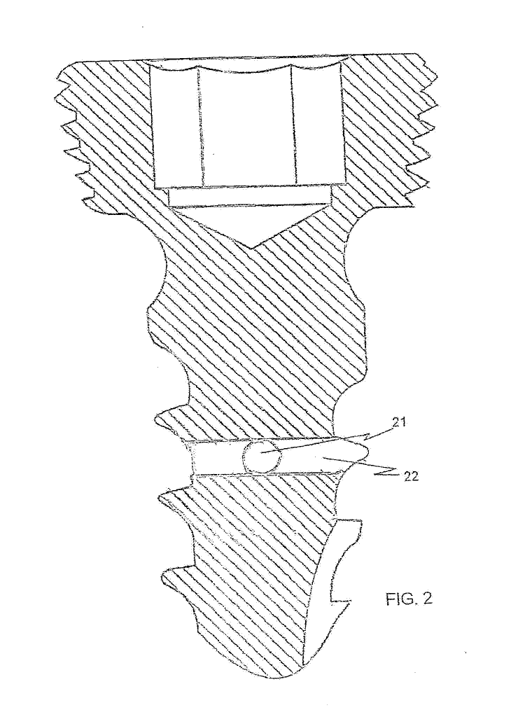

Further, as illustrated in FIG. 1, the shaft has at least one hollow portion in the form of a hole 16 in the shaft. The hole may be preexisting in the material of construction of the bone fastener before the shaft is fabricated, or the hole may be drilled or carved after the shaft is fabricated. In the embodiment illustrated in FIG. 2, two holes 21, 22 are perpendicular to each other and to the axis of the shaft. In another embodiment, the at least one hole may cross the full width or diameter of the shaft, or may be partial and terminating in the body of the shaft. The hole may be perpendicular or angular to the axis of the shaft. The at least one hole may be partially or completely filled with materials having properties that promote fibrous growth, healing or treatment of diseases. The filling of the materials may be done before the fastener is inserted in the bone. The perimeter of the hole may have sharp e edges that will facilitate the cutting of tissue when the fastener is rotated. The sharp edges enhance the ability to remove the fasteners even after the body tissue has grown inside the porosities and the hollow portion of the fastener.

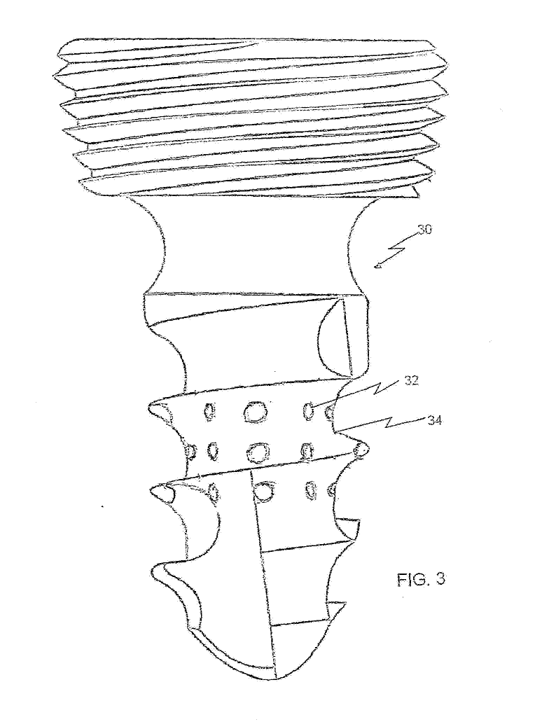

FIG. 3 illustrates in a side view another embodiment of bone fastener 30 having at least one hollow portion in the form of a cavity 32 on the side 34 of the fastener. The cavities can be straight (e.g., cylindrical), curved (e.g., spherical), or tapering (conical). The depth of the cavities can vary from about 0.1 mm to about 10 mm The cavity openings can be of various shapes such as round, square, rectangular, triangular, trapezoidal, and polygonal. The openings can have width, diameter or one of its sides varying from about 0.1 mm to about the diameter of the shaft of the fastener. The at least one cavity may be partially or completely filled with materials having properties that promote fibrous growth, healing or treatment of diseases.

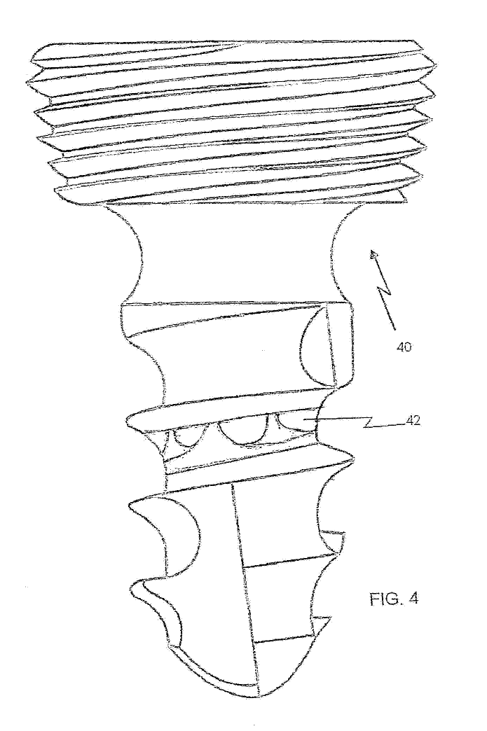

FIG. 4 illustrates in a side view another embodiment of bone fastener 40 wherein a part of the shaft is carved to provide hollow portion and openings 42 in the shaft. The hollow portion may be partially or completely filled with materials having properties that promote fibrous growth, healing or treatment of diseases. The fibrous growth in the hollow portion enhances the securing of the bone fastener to the bone

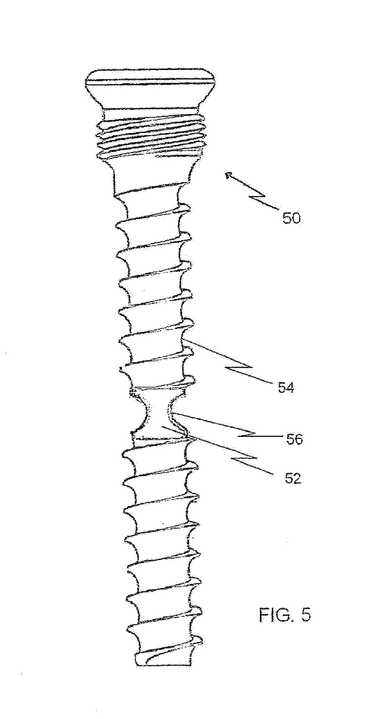

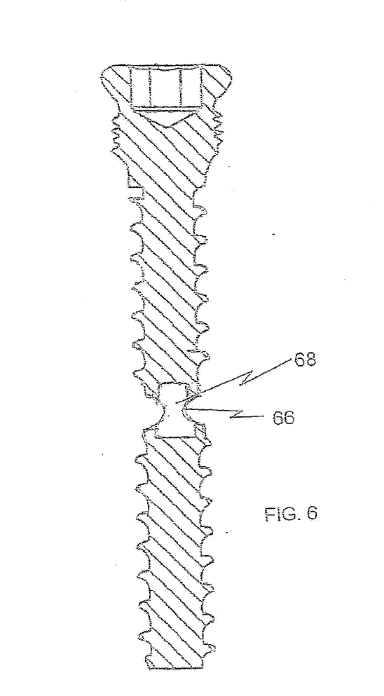

FIG. 5 shows a side view of a bone fastener 50 according to another embodiment. A part 52 of the shaft 54 of fastener 50 is not threaded and has at least one opening 56 communicating with a hollow portion inside the fastener. FIG. 6 is a cross sectional view of the embodiment of bone fastener illustrated in FIG. 5 and shows opening 66 communicating with the hollow portion 68. In another embodiment, the entire length of the shaft may not be threaded and a hollow portion provided inside the shaft with at least one opening on the side of the shaft communicating with the hollow portion. In a further embodiment, multiple hollow portions may be present inside the shaft, with each hollow portion communicating with the outside of the shaft through at least one opening.

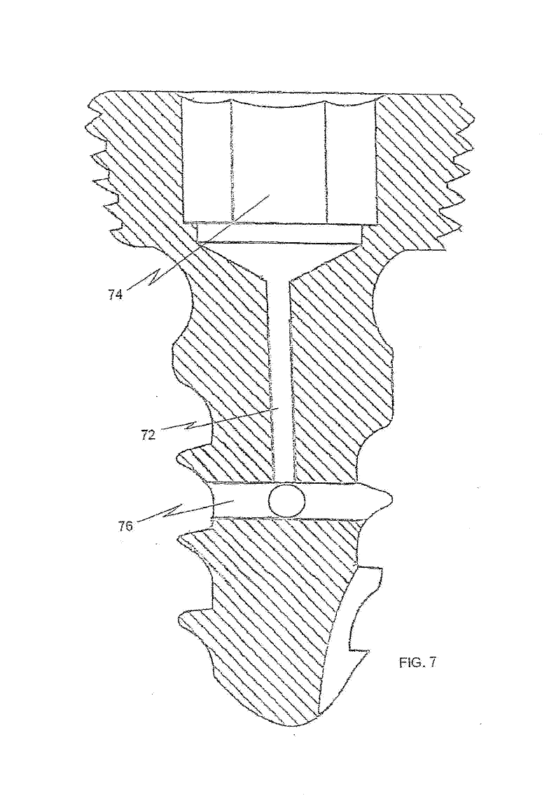

FIG. 7 illustrates in a cross-sectional view an embodiment of the bone fastener shown in FIG. 1 and having a channel 72 connecting the head 74 to the hollow portion 76. The channel may be used to introduce materials in the hollow portion, e.g., by injection from the head 74 or to remove materials from the hollow portion, e. g., by suction at the head 74. The channel allows access to the hollow portion after the fastener is inserted in the bone. The materials can have properties that will promote fibrous growth, healing or treatment of diseases.

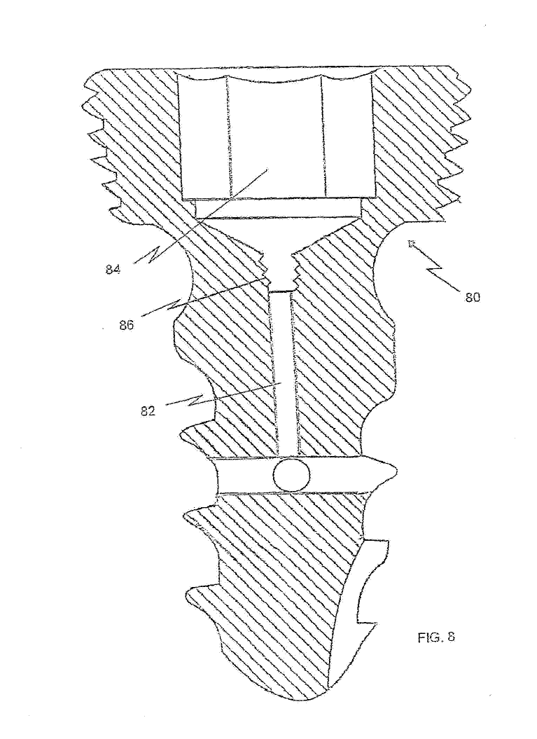

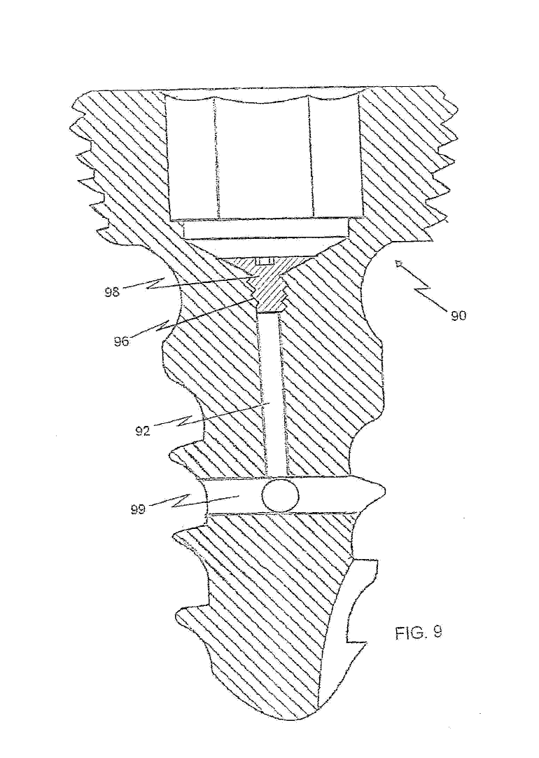

FIG. 8 illustrates a cross-sectional view of another embodiment of bone fastener 80, having a threaded portion 86 to accept a plug to the channel 82 at the head 84. FIG. 9 illustrates a cross-sectional view of an embodiment of bone fastener 90 wherein a plug 98 engages the thread 96 at the end of channel 92 to seal the channel. The plug 98 prevents the transmission of materials from inside the hollow portion 99 and channel 92 to a space outside of the bone after the bone fastener 90 is inserted in a bone.

What is claimed is:

1. A bone fastener for use in orthopedic surgery, comprising a shaft configured to engage bone, said shaft having a hollow portion inside the shaft and at least one opening on the outside of the shaft communicating with the hollow portion.

2. The bone fastener according to claim 1, further comprising a head at distal end of the shaft and a channel connecting the head to the hollow portion.

3. The bone fastener according to claim 1, wherein the hollow portion contains material that promotes fibrous growth, healing and/or treatment of diseases.

4. The bone fastener according to claim 3, wherein the material that promotes fibrous growth, healing and/or treatment of diseases comprises at least one selected from the group consisting of collagen, insoluble collagen derivatives and soluble solids and/or liquids dissolved therein, e.g., antiviral agents, particularly those effective against HIV and hepatitis; antimicrobials and/or antibiotics such as erythromycin, bacitracin, neomycin, penicillin, polymyxin B, tetracyclines, chloromycetin and streptomycins, cefazolin, vancomycin, ampicillin, azactam, tobramycin, clindamycin, and gentamicin; amino acids; peptides; vitamins; inorganic elements; co-factors for protein synthesis; endocrine tissue or tissue fragments, synthesizers; enzymes such as collagenase, peptidases, oxidases; polymer cell scaffolds with parenchymal cells; angiogenic drugs and polymeric carriers containing such drugs; collagen lattices; antigenic agents; cartilage fragments, living cells such as chondrocytes, mesenchymal stem cells; natural extracts; genetically engineered living cells or otherwise modified living cells; tissue transplants; bone morphogenic protein; anti-neoplastic agents; DNA delivered by plasmid or viral vectors; autogenous tissues such as blood, serum, soft tissue, bone marrow; bioadhesives; fibronectin; transforming growth factor-beta; endothelial cell growth factors; cementum attachment extracts; ketaserin; insulin-like growth factors (IGF-1)(IGF-2); platelet derived growth factors (PDGF); epidermal growth factor (EGF); interleukin; human alphathrombin; fibroblast growth factors; hormones, in particular, human growth hormone, animal growth hormone and growth hormones such as somatotropin; bone digesters; antitumor agents; immuno-suppressants; permeation enhancers, e.g., fatty acid ester such as laureate, myristate and stearate monoesters of polyethylene glycol, enamine derivatives, alpha-keto aldehydes; and, nucleic acids.

5. The bone fastener according to claim 2, wherein the hollow portion contains material that promotes fibrous growth, healing and/or treatment of diseases.

6. The bone fastener according to claim 5, wherein the material that promotes fibrous growth, healing and/or treatment of diseases comprises at least one selected from the group consisting of collagen, insoluble collagen derivatives and soluble solids and/or liquids dissolved therein, e.g., antiviral agents, particularly those effective against HIV and hepatitis; antimicrobials and/or antibiotics such as erythromycin, bacitracin, neomycin, penicillin, polymyxin B, tetracyclines, chloromycetin and streptomycins, cefazolin, vancomycin, ampicillin, azactam, tobramycin, clindamycin, and gentamicin; amino acids; peptides; vitamins; inorganic elements; co-factors for protein synthesis; endocrine tissue or tissue fragments, synthesizers; enzymes such as collagenase, peptidases, oxidases; polymer cell scaffolds with parenchymal cells; angiogenic drugs and polymeric carriers containing such drugs; collagen lattices; antigenic agents; cartilage fragments, living cells such as chondrocytes, mesenchymal stem cells; natural extracts; genetically engineered living cells or otherwise modified living cells; tissue transplants; bone morphogenic protein; anti-neoplastic agents; DNA delivered by plasmid or viral vectors; autogenous tissues such as blood, serum, soft tissue, bone marrow; bioadhesives; fibronectin; transforming growth factor-beta; endothelial cell growth factors; cementum attachment extracts; ketaserin; insulin-like growth factors (IGF-1)(IGF-2); platelet derived growth factors (PDGF); epidermal growth factor (EGF); interleukin; human alphathrombin; fibroblast growth factors; hormones, in particular, human growth hormone, animal growth hormone and growth hormones such as somatotropin; bone digesters; antitumor agents; immuno-suppressants; permeation enhancers, e.g., fatty acid ester such as laureate, myristate and stearate monoesters of polyethylene glycol, enamine derivatives, alpha-keto aldehydes; and, nucleic acids.

7. The bone fastener according to claim 2, further comprising a plug to seal the channel at the head.

8. The bone fastener according to claim 1, wherein the at least one opening further comprises sharp edges at the perimeter of the opening facilitating the cutting of tissue when the fastener is rotated.

9. The bone fastener according to claim 3, wherein the materials are included in the hollow portion before the fastener is inserted in the bone.

10. The bone fastener according to claim 5, wherein the materials are introduced in the hollow portion through the channel after the fastener is inserted in the bone.

11. The bone fastener according to claim 1, wherein the hollow portion inside the shaft is at least one hole drilled or carved in the shaft,

12. A method for treating diseases, the method comprising:
inserting a shaft of a bone fastener in a bone;
the bone fastener comprising a hollow portion inside the shaft and at least one opening on the outside of the shaft communicating with the hollow portion;
wherein the hollow portion contains materials that promote treatment of diseases.

13. The method for treating diseases according to claim 12, further comprising:
a head at distal end of the shaft and a channel connecting the head to the hollow portion; and
introducing the materials from the head into the hollow portion through the channel.

14. The method for treating diseases according to claim 13, further comprising a plug to seal the channel.

15. A bone fastener for use in orthopedic surgery, comprising a shaft configured to engage bone, said shaft having at least one hollow portion in the form of a cavity on the side of the fastener.

16. The bone fastener according to claim 15, wherein the cavity is cylindrical, spherical or conical.

17. The bone fastener according to claim 15, wherein at least one cavity may be partially or completely filled with material that promotes fibrous growth, healing and/or treatment of diseases.

18. The bone fastener according to claim 17, wherein the material that promotes fibrous growth, healing and/or treatment of diseases comprises at least one selected from the group consisting of collagen, insoluble collagen derivatives and soluble solids and/or liquids dissolved therein, e.g., antiviral agents, particularly those effective against HIV and hepatitis; antimicrobials and/or antibiotics such as erythromycin, bacitracin, neomycin, penicillin, polymyxin B, tetracyclines, chloromycetin and streptomycins, cefazolin, vancomycin, ampicillin, azactam, tobramycin, clindamycin, and gentamicin; amino acids; peptides; vitamins; inorganic elements; co-factors for protein synthesis; endocrine tissue or tissue fragments, synthesizers; enzymes such as collagenase, peptidases, oxidases; polymer cell scaffolds with parenchymal cells; angiogenic drugs and polymeric carriers containing such drugs; collagen lattices; antigenic agents; cartilage fragments, living cells such as chondrocytes, mesenchymal stem cells; natural extracts; genetically engineered living cells or otherwise modified living cells; tissue transplants; bone morphogenic protein; anti-neoplastic agents; DNA delivered by plasmid or viral vectors; autogenous tissues such as blood, serum, soft tissue, bone marrow; bioadhesives; fibronectin; transforming growth factor-beta; endothelial cell growth factors; cementum attachment extracts; ketaserin; insulin-like growth factors (IGF-1)(IGF-2); platelet derived growth factors (PDGF); epidermal growth factor (EGF); interleukin; human alphathrombin; fibroblast growth factors; hormones, in particular, human growth hormone, animal growth hormone and growth hormones such as somatotropin; bone digesters; antitumor agents; immuno-suppressants; permeation enhancers, e.g., fatty acid ester such as laureate, myristate and stearate monoesters of polyethylene glycol, enamine derivatives, alpha-keto aldehydes; and, nucleic acids.

19. The bone fastener according to claim 16, wherein the depth of the cavity is in the range of about 0.1 mm to about 10 mm.

20. The bone fastener according to claim 17, wherein the depth of the cavity is in the range of about 0.1 mm to about 10 mm.
